(19) United States
(12) Patent Application Publication
(10) Pub. No.: US 2011/0093002 A1
(43) Pub. Date: Apr. 21, 2011

(54) STENT-WITHIN-STENT ARRANGEMENTS

(75) Inventors: Brian K. Rucker, King, NC (US); Caroline M. Gayzik, Winston-Salem, NC (US)

(73) Assignee: Wilson-Cook Medical Inc., Winston-Salem, NC (US)

(21) Appl. No.: 12/582,286

(22) Filed: Oct. 20, 2009

(51) Int. Cl. A61M 29/00; A61F 2/04

(52) U.S. Cl. 606/198; 623/23.7

(57) ABSTRACT

A variety of stent arrangements are described in which multiple stents expand and coordinate to block the spaces between the struts of the outer stent to create a tubular stent not prone to tissue in-growth. One or more stents are selectively positioned within an outer stent such that the struts of the one or more stents at least partially fill the openings of the outer stent. Alternatively, the one or more stents may be permanently affixed to the outer stent to produce a stent arrangement in which the openings between the struts of the outer stent are blocked by the struts of the one or more stents.

### BACKGROUND

The present invention relates generally to medical devices and more particularly to stent arrangements that are used to dilate narrowed portions of a body lumen.

Stents are widely used in the medical profession to enlarge, dilate or maintain the patency of narrowed body lumens. A stent may be positioned across a narrowed region while the stent is in a compressed state. The stent may then be expanded in order to widen the lumen.

Stents used in the gastrointestinal system have been typically constructed of plastic. Plastic stents facilitate retrieval and/or replacement of the stent during a follow-up procedure. However, plastic stents are not expandable, thereby possessing a fixed diameter. Since plastic stents are frequently delivered through the working channel of an endoscope, the diameter of the working channel limits the diameter of the stent. For example, plastic stents typically have a diameter that is no greater than 11.5 French. However, such a small diameter stent rapidly becomes clogged within the biliary and pancreatic ducts, thereby requiring replacement every three months, or even sooner.

Stents constructed of various metal alloys have also been used within the biliary and pancreatic ducts. These types of metal stents may be self-expanding or balloon expandable, and are designed to expand to a much larger diameter than the plastic stents described above. Consequently, such metal stents remain patent longer than plastic stents, averaging perhaps 6 months before clogging. However, the capability of larger diameter stents to collapse into endoscopic delivery systems necessitates mesh or wire geometries that incur tissue in-growth, commonly known as endothelialization, thereby oftentimes rendering the stent permanent and impossible to remove. Therefore, even when a retrievable metal stent has been employed, it may not be possible to remove it without damaging surrounding tissues.

In view of the drawbacks of current stents, an improved stent is needed that limits endothelialization. Although the inventions described below may be useful in limiting endothelialization, the claimed inventions may solve other problems as well.

### SUMMARY

Accordingly, a stent-within-a-stent arrangement is provided to address the above-described drawbacks.

In a first aspect, a medical device for dilation of a body lumen is provided. A medical device for dilation of a body lumen comprises an expandable outer prosthesis formed from a plurality of outer struts, in which each of the plurality of outer struts is spaced apart to form outer openings therebetween. An expandable inner prosthesis is formed from a plurality of inner struts, in which each of the plurality of inner struts is spaced apart to form a plurality of inner openings therebetween. The inner prosthesis is disposed within a portion of a lumen of the outer prosthesis so that a portion of the inner struts at least partially block the outer openings.

In a second aspect, a medical device for dilation of a body lumen is provided. The device comprises an outer stent comprising outer struts spaced apart to form outer spaces therebetween. An inner stent is also provided. The inner stent comprises inner struts spaced apart to form inner spaces therebetween. At least a portion of the inner stent is slidably interfitted within the outer stent. An interlocking element fixates the inner stent within the outer stent. At least a portion of the inner struts occupy the outer spaces of the outer struts to substantially prevent tissue in-growth therethrough.

In a third aspect, a method of implanting a stent arrangement into a body lumen is provided comprising the following steps. An outer stent and an inner stent are delivered to the body lumen. The outer stent and the inner stent are deployed at a target site within the body lumen. The outer stent expands from a first diameter to a second diameter greater than the first diameter. The outer stent has a plurality of outer struts spaced apart at the second diameter to form a plurality of outer openings. The inner stent is then interlocked to the outer stent.

### BRIEF DESCRIPTION OF SEVERAL VIEWS OF THE DRAWINGS

The invention may be more fully understood by reading the following description in conjunction with the drawings, in which:

FIG. 1a is a side view of a compressed stent that is to be deployed and anchored within an outer stent;

FIG. 1b is a side view of the outer stent shown in its expanded state into which the compressed stent of FIG. 1b is to be deployed therewithin;

FIG. 2 is a perspective view of the compressed stent of FIG. 1a expanded and anchored within the outer stent of FIG. 1b;

FIG. 3 is a cross-sectional view of FIG. 2 showing the anchors affixed to the inner stent and extending through the interstices of the outer stent to interlock the inner stent to the outer stent;

FIG. 4 is a side view of an inner stent anchored within an outer stent within a stenosed region of a body lumen;

FIG. 5a is a side view of a compressed inner stent that is to be deployed and anchored within an outer stent;

FIG. 5b is a side view of the outer stent shown in its expanded state into which the compressed stent of FIG. 5a is to be deployed therewithin;

FIG. 6 is a perspective view of the compressed stent of FIG. 5a expanded and anchored within the outer stent of FIG. 5b to create a stent-within-stent arrangement;

FIG. 7a shows a partial cross-sectional view through walls of an outer z-stent;

FIG. 7b shows a partial cross-sectional view through walls of an inner z-stent disposed slightly offset from the outer z-stent of FIG. 7a to create a stent-within-stent arrangement in which the struts of the inner z-stent occupy the interstices of the outer z-stent;

FIG. 8 shows an end view of inwardly bent crowns of an outer braided stent engaging with struts of an inner stent;

FIG. 9 is a side view of FIG. 8;

FIG. 10 is a cross-sectional view of an inner stent permanently affixed at its distal end to an outer stent by shape memory spacer bars in which the stent pattern of the inner and outer stents coincide or align with each other;

FIG. 11 is a cross-sectional view of the stent-within-stent arrangement of FIG. 10 in which the spacer bars have been activated to shift the inner stent a predetermined distance such that the outer mesh openings of the outer stent are at least partially covered or blocked by the inner struts of the inner stent;

FIG. 12 shows a cross sectional view of a braided stent that contains a removable inner sleeve disposed within the lumen and along the interior surface of the outer stent;

FIG. 13 shows an embodiment in which an expanded coiled inner stent is disposed within the lumen of an expanded outer z-stent;

FIG. 14 shows an inner strut of an inner stent and an outer strut of an outer stent coupled to each other with a cannula to create a single coupling point;

FIG. 15 shows the holes of the inner stent and the outer stent aligned with each other at each of their respective distal ends;

FIG. 16 shows an inner stent magnetically coupled to an outer stent

FIG. 17 shows a stent-within-stent arrangement in which an inner stent is welded to an outer stent;

FIG. 18 shows a cross-sectional view of a single introducer loaded with an inner stent and an outer stent; and

FIG. 19 shows an alternative delivery introducer serially loaded with a first stent and a second stent spaced apart proximally from the first stent.

### DETAILED DESCRIPTION

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details, which are not necessary for an understanding of the present invention, have been omitted such as conventional details of fabrication and assembly.

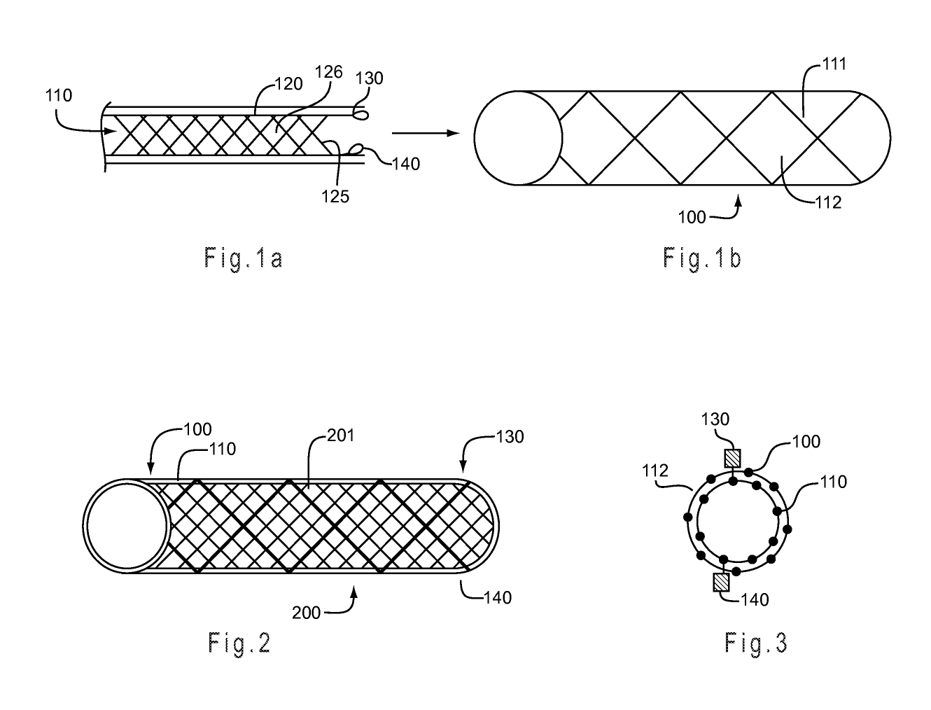

FIG. 1a illustrates a side view of an inner stent 110 that is to be deployed and anchored within an outer stent 100. The outer stent 100 is shown in FIG. 1b as deployed and in its expanded state. The outer stent 100 has struts 111 which create a mesh design. The struts 111 are spaced apart in the expanded state so as to create interstices 112 (i.e., meshed openings defined by adjacent struts). The inner stent 110 is shown constrained within a retractable outer delivery sheath 120 of a delivery catheter. FIG. 1a shows that the inner stent 110 has struts 125 which also create a mesh design. As shown in FIGS. 1a and 1b, the mesh design of the inner stent 110 may have a greater helical pitch (i.e., a tighter weave) than that of outer stent 100. Anchors 130 and 140 are shown affixed to the distal end of the inner stent 110. The anchors 130 and 140 act as coupling members for coupling the inner stent 110 with the outer stent 100. Preferably, the anchors 130 and 140 as shown in FIG. 1a are substantially parallel to the longitudinal axis of the outer delivery sheath 120 to ensure that the anchors 130 and 140 minimize frictional resistance during proximal retraction of the outer delivery sheath 120. Additionally, the parallel orientation of the anchors 130 and 140 maintains a sufficiently small profile of the outer delivery sheath 120 and the inner stent 110 during delivery into the lumen of the expanded outer stent 100. Alternatively, the anchors 130 and 140 may be angled inwards during delivery.

Generally speaking, the anchors 130 and 140 act to interlock the inner stent 110 with the outer stent 100 as the inner stent 110 becomes deployed within the lumen of the outer stent 100. In other words, the anchors 130 and 140 function as coupling or engagement members to couple/engage the inner stent 110 with the outer stent 100. When in the deployed configuration, the struts 125 of the deployed inner stent 110 are disposed so as to cover or overlie the interstices 112 of the outer stent 100. The net result is that at least a fraction of the interstices 112 are blocked by the inner stent 110, thereby reducing the effective or resultant free space between the struts 111 of the outer stent 100. Such a reduction in free space between the struts 111 of the outer stent 100 may significantly reduce tissue ingrowth through the struts 111 of the outer stent 100. When the inner stent 110 interlocks with the outer stent 100 as shown in FIG. 2, the anchors 130 and 140 move from their parallel orientation as shown in FIG. 1a into an outward direction as shown in FIG. 2. Such movement may occur because of shape memory properties possessed by the anchors 130 and 140. As the anchors 130, 140 move outwards to the second position, they extend through the interstices 112 of outer stent 100 and thereafter catch on the struts 111 of the outer stent 100. The anchors 130 and 140 function to secure the inner stent 110 to the outer stent 100. This anchored position prevents the inner stent 110 from sliding out of outer stent 100. Although the anchors 130, 140 are shown positioned at the distal end of inner stent 110, the anchors 130, 140 may also be positioned at the proximal end of the inner stent 110 and/or at various predetermined locations along the inner stent 110. Although two anchors 130, 140 are shown, one anchor or more than two anchors may optionally be used.

FIG. 2 shows the inner stent 110 completely deployed within the outer stent 100 to produce a stent-within-a-stent arrangement 200. The inner stent 110 may have any diameter. The inner stent 110 may be the same diameter as the outer stent 100. Alternatively, the inner stent 100 may have a larger diameter than the outer stent 100 to ensure that the inner stent 100 expands tightly against the interior surface of the outer stent 100. Generally speaking, an inner stent 110 that has the same diameter or a larger diameter than that of the outer stent 100 will, upon expansion, exert an outwardly directed radial force against the inner surface of the outer stent 100 that is sufficient in creating and maintaining an adequate fit between the stents 100, 110, as discussed below in connection with FIG. 3. The contribution of an outward radial force by inner stent 110 may also assist in maintaining the stent-within-stent arrangement 200 fixated at the target site.

FIG. 3 is a cross-sectional view of the stent-within-a-stent arrangement 200 of FIG. 2. FIG. 3 shows that the inner stent 110 has radially expanded against the inner surface of outer stent 100, with anchors 130 and 140 having moved from the parallel orientation to the outwardly bent orientation through mesh openings 112 of the outer stent 100, thereby interlocking the inner stent 110 to the struts 111 of the outer stent 100.

As previously noted, FIG. 2 shows that the tighter weave of the inner stent 110 substantially fills the mesh openings of the outer stent 100. The resultant mesh openings 201 of the stent-within-a-stent 200 arrangement are shown to be significantly smaller than the mesh openings 112 of stent 100. As a result of the smaller mesh openings 201, the stent-within-a-stent 200 may not be susceptible to significant tissue in-growth when implanted in a body lumen.

Although not shown in FIG. 2, a third stent may be inserted within the inner stent 110 to further reduce the mesh openings of the outer stent 100. The third stent may have a tighter weave pattern than the outer stent 100 or inner stent 110 in order for its struts to further occupy the mesh openings 201. Alternatively, if the third stent has the same weave pattern as the outer stent 100, the third stent may be selectively offset from the outer stent 100 such that its struts may block the mesh openings. Two or more stents may be needed to substantially block the mesh openings when the stents have a large fraction of free space relative to struts. The exact number of stents to be deployed within each other may depend, at least in part, on the size of the body lumen and the degree of tissue ingrowth desired to be prevented.

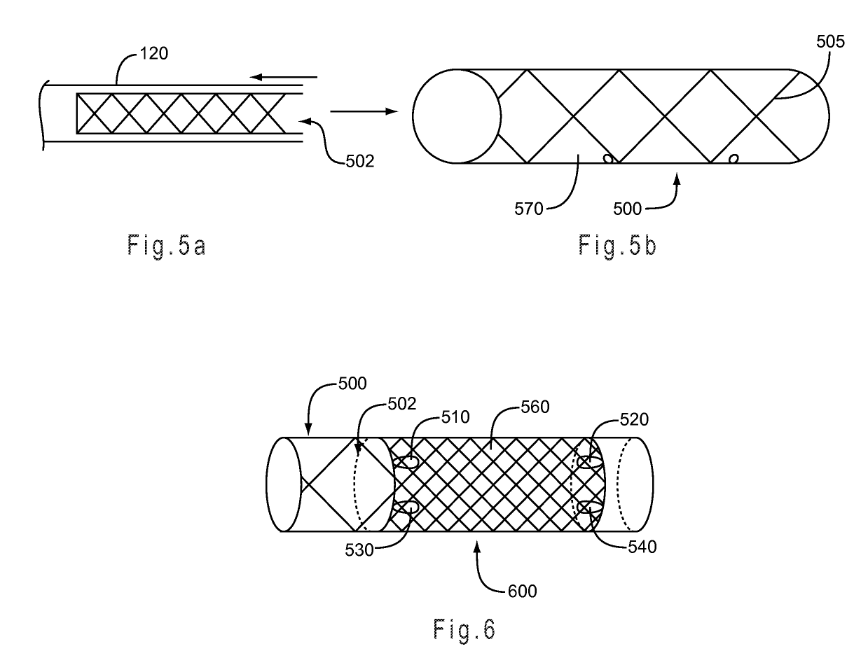

Although FIG. 2 shows the inner stent 110 having the same longitudinal length as the outer stent 100 such that all of the mesh openings 112 of the outer stent 100 are filled by the struts 125 of the inner stent 110, inner stent 110 may be shorter in length than the outer stent 100 to produce a stent-within-a-stent 600 as shown in FIG. 6. FIG. 6 shows an inner stent 502 within an outer stent 500. The inner stent 502 is shorter in length than the outer stent 500. Unlike the embodiment of FIGS. 1a-3, the outer stent 500 has anchors 510, 520, 530, 540. The anchors 510, 520, 530, 540 are initially parallel to the longitudinal axis of the outer stent 500, as shown in FIG. 5. Upon deployment and expansion of the inner stent 502 within the outer stent 500, the anchors 510, 520, 530, 540 move to the position shown in FIG. 6. The anchors 510, 520, 530, 540 move inwards through the interstices of the inner stent 502 and thereafter catch on the struts of the inner stent 502. This anchorage prevents the inner stent 502 from sliding out of outer stent 500.

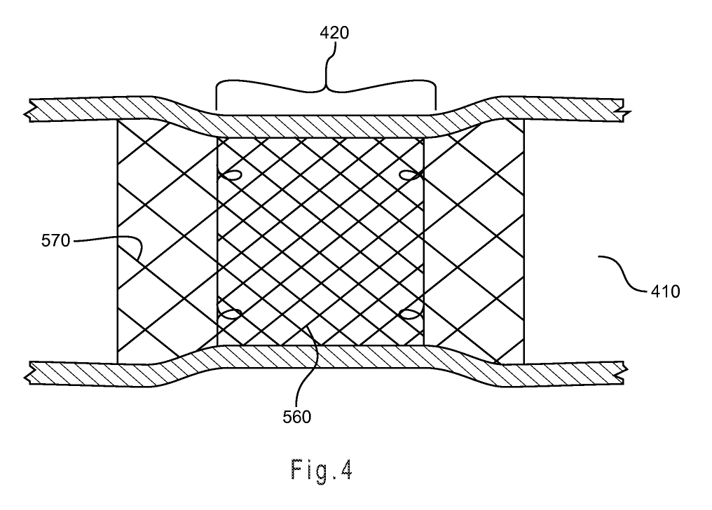

The inner stent 502 is slidably interfitted within the central portion of the outer stent 500 to produce a stent-within-a-stent 600 which contains mesh openings 560 that are smaller than the mesh openings 570 (FIG. 5) of outer stent 500. The end portions of the stent-within-a-stent 600 possess mesh openings 570 of the outer stent 500. As FIG. 4 shows, the stent-within-a-stent 600 of FIG. 6 may be implanted in a body lumen 410 such that the stenosed region 420 aligns with the smaller mesh openings 560. The mesh openings 560 would be sufficiently small such that significant tissue in-growth may be prevented therethrough. The larger mesh openings 570 at the end portions of the stent-within-a-stent 600 extend along the unstenosed portions of the body lumen 410. Thus, tissue in-growth would occur through the larger mesh openings 570, which is favorable because it allows the stent-within-a-stent 600 to be sufficiently anchored within the body lumen 410.

FIGS. 1-6 have shown an inner stent 110 with a tighter weave pattern that is slidably interfitted and aligned within the outer stent 100 such that the struts 125 of the inner stent 110 occupy and block the mesh openings 112 of the outer stent 100 to prevent tissue-in growth. As an alternative, the weave pattern of the inner stent 110 need not be tighter than that of the outer stent 100. Rather, the weave pattern of the inner stent 110 could be the same as that of the outer stent 100. When deploying the inner stent 110 within the outer stent 100, the outer sheath 120 of delivery catheter would deploy the inner stent 110 within the lumen of the outer stent 100 at a selectively offset position relative to the outer stent 100 such that the struts 125 of the inner stent 110 would occupy the mesh openings 112 of the outer stent.

Various stent architectures can be used to create the stent-within stent arrangements, including, but not limited to, braided, zig-zag, laser cut, and serpentine configurations. Generally speaking, the stents can include any type of expandable member having solid members with openings therebetween.

Additionally, although all of the Figures have illustrated the inner and outer stents to have the same stent architecture, the inner and outer stents can have different stent architectures. For example, the outer stent could comprise a stent pattern having a high fraction of free interstitial spaces relative to struts. Accordingly, the inner stent would have a suitable stent architecture that contains less free space relative to that of the outer stent, thereby enabling the struts of the inner stent to be disposed so as to cover or block the free spaces of the outer stent.

Preferably, the anchors that have been described are made from a shape memory material, such as nitinol. A shape memory material may undergo a substantially reversible phase transformation that allows it to “remember” and return to a previous shape or configuration. For example, in the case of nickel-titanium alloys, a transformation between an austenitic phase and a martensitic phase may occur by cooling and/or heating (shape memory effect) or by isothermally applying and/or removing stress (superelastic effect). Austenite is characteristically the stronger phase (i.e., greater tensile strength) and martensite is the more easily deformable phase. In an example of the shape memory effect, a nickel-titanium alloy having an initial configuration in the austenitic phase may be cooled below a transformation temperature (M_{f}) to the martensitic phase and then deformed to a second configuration. Upon heating to another transformation temperature (A_{f}), the material may spontaneously return to its initial configuration. Generally, the memory effect is one-way, which means that the spontaneous change from one configuration to another occurs only upon heating. However, it is possible to obtain a two-way shape memory effect, in which a shape memory material spontaneously changes shape upon cooling as well as upon heating.

Applying the shape memory effect principles described, the nitinol anchors would be made at a transformation temperature in which the anchors are heat set to the interlocking configuration (e.g., FIGS. 2, 4, and 6). Preferably, the temperature at which the nitinol would be made would be slightly below about body temperature. Hence, when the anchors are being delivered to the target site of a body lumen, the anchors are below the transformation temperature thereby possessing the martensitic crystal phase in which the anchors can be readily compressed and manipulated to the desired parallel configuration (FIGS. 1 and 5). Preferably, the anchors are not bent outwardly during delivery to avoid the anchors scraping the surface of the delivery sheath of the catheter. Thus, preferably, the anchors are configured such that they are flush with the delivery catheter 120. Alternatively, the anchors may be configured such that they are angled inwards. Upon the inner stent being partially deployed within the outer stent, the nitinol anchors would be heat activated so that they return to their original, manufactured shape (i.e., the “remembered” austenitic state) in which the anchors are bent outwards. For example, warm water could be injected over the surface of the anchors. The temperature of the warm water would be slightly greater than body temperature to cause the anchors to move from their compressed, deformed configuration during delivery (i.e., the martensitic phase) to their interlocking, bent outwards configuration during deployment (i.e., the austenitic phase). The temperature of the warm water would not be as high as boiling because the tissue would be damaged.

As an alternative to heat activation of a shape memory alloy, pressure activation may be utilized to revert the anchors from the deformed configuration during delivery to the inwardly bent shape (if anchors are affixed to outer stent) or the outwardly bent shape (if anchors are affixed to inner stent) during deployment. A stress-induced martensite (SIM) alloy may be used in which the superelastic effect is utilized. This involves applying stress to a shape memory material having an initial shape in the austenitic phase to cause a transformation to the martensitic phase without a change in temperature. A return transformation to the austenitic phase may be achieved by removing the applied stress. The superelastic effect may be exploited at a temperature above A_{f}. However, if the temperature is raised beyond a temperature of M_{d}, which may be about 50° C. above A_{f}, the applied stress may plastically (permanently) deform the austenitic phase instead of inducing the formation of martensite. In this case, not all of the deformation may be recovered when the stress is removed. Suitable alloys displaying SIM at temperatures near body temperature may be selected from known shape memory alloys by those of ordinary skill in the art.

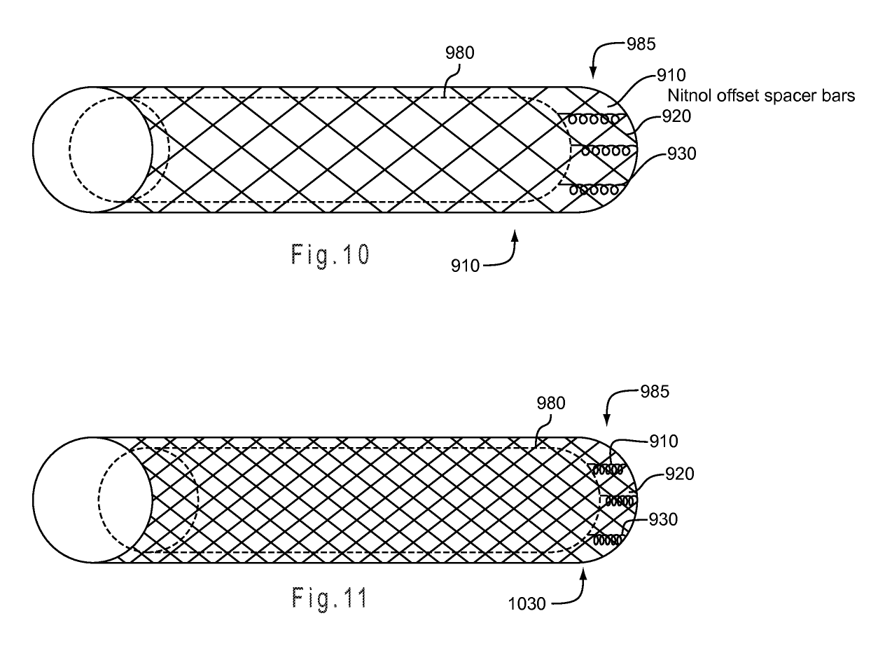

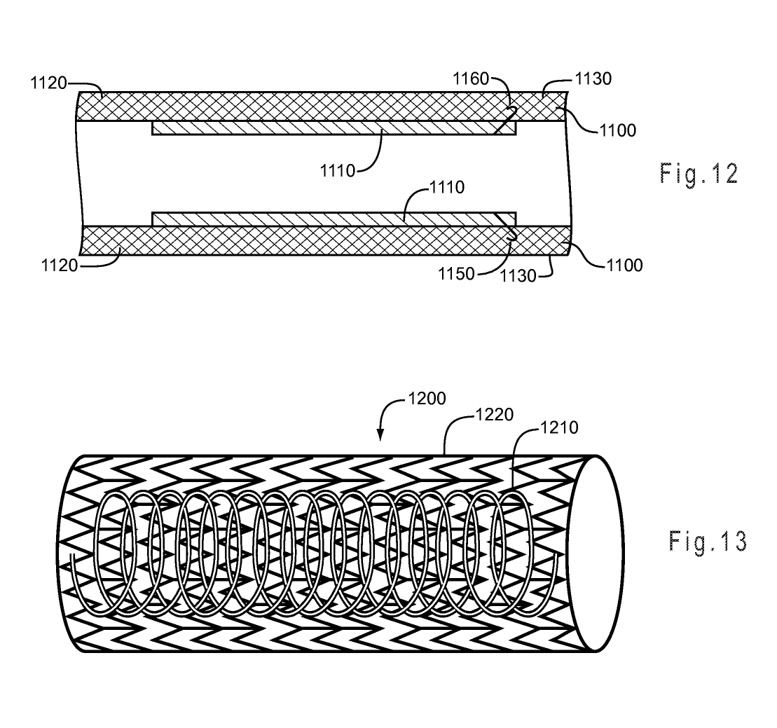

The above embodiments have discussed stent-within-a-stent arrangements in which the inner and outer stents are deployed separately. Stent-within-a-stent arrangements in which the inner stent is permanently affixed to the outer stent are also contemplated. FIGS. 10 and 11 show an inner stent 980 that is permanently affixed to the outer stent 985 by shape memory spacer bars 910, 920, and 930. In one embodiment, the spacer bars 910, 920, 930 are formed from a nickel-titanium alloy such as nitinol. The nitinol spacer bars 910, 920, 930 connect the distal end of the inner stent 980 with the distal end of the outer stent 985. The spacer bars 910, 920, 930 possess spring-like properties. Upon heat activation of the nitinol spacer bars, the spacer bars 910, 920, 930 can compress, thereby shifting the inner stent 980 relative to the outer stent 985. FIG. 10 shows that the spacer bars 910, 920, and 930 are configured such that the struts of the inner stent 980 coincide with the struts of the outer stent 985. Because the inner stent 980 is aligned with the outer stent 985, they may be sufficiently constrained together within a delivery catheter. After the stent arrangement 900 of FIG. 10 has been delivered to the target site and the inner and outer stents 980, 985 have been allowed to radially expand, the nitinol spacer bars 910, 920, 930 may be heat activated, as known in the art, to shift the inner stent 980 distally as shown in FIG. 11. When the spacer bars 910, 920, 930 are heat activated (e.g., by injection of warm water onto the spacer bars 910, 920, 930), they shorten a predetermined amount, reverting to their initial compressed position, as shown in FIG. 11. The shortening of the spacer bars 910, 920, 930 by a predetermined amount allows the inner stent 980 to shift distally such that the struts of the inner stent 980 block the open meshes of the outer stent 985, as shown in FIG. 11. Because the inner stent 980 has been shifted a predetermined distance, the open meshes of the stent arrangement 1000 of FIG. 11 are significantly smaller than the open meshes of the stent arrangement 900 of FIG. 10. As an alternative to heat activation, the spacer bars 910, 920, 930 may be formed from a SIM alloy that could be pressure activated. Preferably, the inner stent 980 has the same helical pitch as the outer stent 985 so that the stent arrangement 900 may be effectively constrained within a delivery catheter.

Although not shown, a third stent may be affixed to the stent arrangement of FIGS. 10 and 11 to further fill the mesh openings. The distal end of the third stent could be affixed to the distal end of the outermost stent 980 by a separate set of nitinol spacer bars, which would be designed to compress a certain amount such that the third stent is sufficiently offset relative to the outermost stent 980 and middle stent 985 to further reduce the mesh openings. Numerous factors determine the number of stents that are affixed to each other, as shown in FIG. 10, including the ability of the stents to be constrained within a delivery catheter during delivery and the size of the mesh openings. Generally speaking, a greater number of permanently affixed stents create smaller mesh openings, thereby making tissue in-growth difficult. However, the greater number of permanently affixed stents creates a larger profile during delivery. One of ordinary skill would understand how to balance these competing factors, along with other factors, in view of the particular application to determine the ideal number of stents to be utilized.

The embodiment of FIGS. 10 and 11 is advantageous in that the inner stent 980 need not be manipulated in order to interlock it with the outer stent 985 and/or block the gaps of the outer stent 985. Rather, and has been described above, the inner and outer stents 980, 985 are already aligned in their proper positions. Subsequent heat or pressure activation of the nitinol spacer bars 910, 920, 930 causes the inner stent 980 to slide a predetermined amount to offset the outer stent 985 at its so-called blocking position.

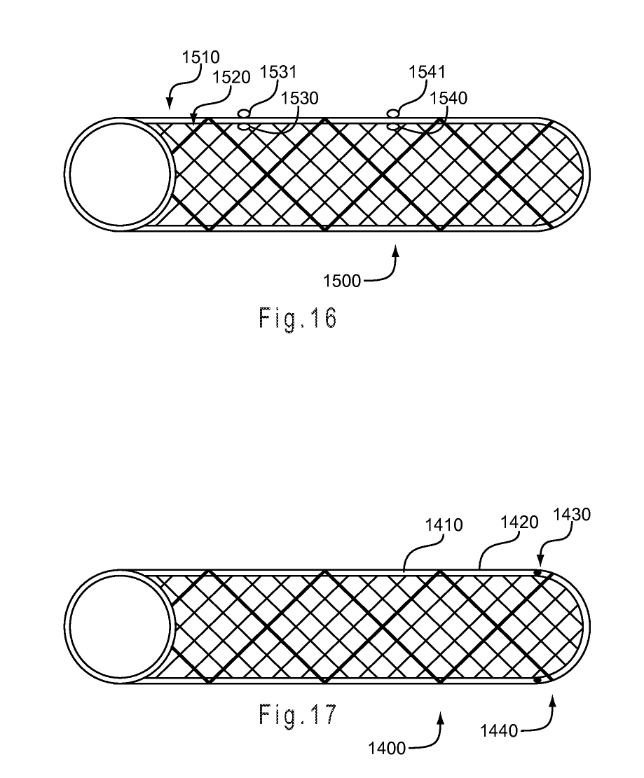

FIGS. 16 and 17 are examples of other ways in which the inner stent may be permanently attached to the outer stent. FIG. 17 illustrates a stent arrangement 1400 in which an inner stent 1410 is welded to an outer stent 1420 at distal points 1430, 1440. FIG. 15 shows inner stent 1520 magnetically coupled to outer stent 1510. In particular, point 1530 on the inner stent 1520 is magnetically coupled to point 1531 of the outer stent 1510, and point 1540 of the inner stent 1520 is magnetically coupled to point 1541 of the outer stent 1510 by placing magnets of opposite polarities at points 1530, 1531 and points 1540, 1541, respectively. The opposite polarities cause the magnets to be magnetically coupled to each other.

The inner stent and outer stent in the embodiments of FIGS. 16 and 17 are affixed such that the open meshes are already in a blocked configuration during delivery. In other words, the inner stents 1520 and 1410 possess a greater helical pitch (i.e., tighter weave) than that of their respective outer stents 1510 and 1420 such that there is no need to offset the inner stents 1520 and 1410 from their respective outer stents 1520 and 1410.

Accordingly, it is preferable to have only one inner stent affixed to the outer stent in order for the stent arrangements 1400 and 1500 to be sufficiently constrained within a delivery catheter. The inner stents 1520 and 1410 of FIGS. 16 and 17 may have the same helical pitch as their respective outer stents 1510 and 1420. If the inner stents 1520 and 1410 do possess the same helical pitch as their respective outer stents 1510 and 1420, then the inner stents 1520 and 1410 are permanently affixed to the outer stents 1510 and 1420 in an offset position relative to their outer stents 1510 an 1420 to allow the struts of the inner stents 1520 and 1410 to block the interstices of the outer stents 1510 and 1420.

Determining whether to utilize a stent-within-a-stent arrangement in which the inner and outer stents are deployed separately or a stent-within-a-stent arrangement in which the inner stent is permanently affixed to the outer stent depends on numerous factors, including the extent to which the stent mesh openings need to be blocked, the target site for implantation, the geometry of the target site, the allowable procedure time, and the profile of the stents when constrained within a delivery catheter. It may be advantageous to utilize a permanently affixed stent-within-a-stent arrangement when the physician does not have time to expend with interlocking the inner stent within the outer stent. Alternatively, it may be advantageous to utilize a stent-within-a-stent arrangement in which the inner and outer stents are deployed separately to achieve greater blockage of mesh openings.

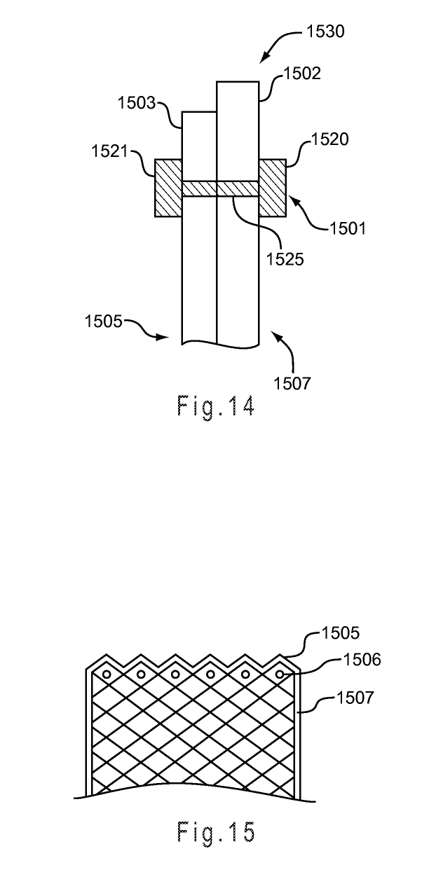

Additional structures and techniques for coupling the inner and outer stents are also contemplated. As an example, FIG. 14 shows that the inner strut 1503 of the inner stent 1505 and the outer strut 1502 of the outer stent 1507 are coupled to each other with a cannula 1501 to create a single coupling point 1530. A hole 1506 extends completely through the inner strut 1503 and the outer strut 1502. The hole 1506 (FIG. 15) is sized so that the body portion 1525 of the cannula 1501 may be inserted completely therethrough. The cannula 1501 has flanged ends 1520 and 1521 which are wider than the hole 1506. Flanged end 1521 abuts against inner strut 1503 and flanged end 1520 abuts against outer strut 1502. The cannula 1501 is preferably a radiopaque marker that enables visualization of the inner and outer stents 1505 and 1507 during deployment. As shown in FIG. 15, the holes 1506 of the inner stent 1505 and the outer stent 1507 may be aligned with each other at each of their respective distal ends to enable insertion of a cannula 1501 therethrough. Coupling of the inner stent 1505 with the outer stent 1507 may involve using an inner stent 1505 that has a different helical pitch (i.e., a greater or lesser helical pitch) than that of the outer stent 1507 so that the interstices of the outer stent 1507 are occupied by the struts of the inner stent 1505. It should be understood that the structures and techniques described for coupling the inner stent 1505 to the outer stent 1507 and positioning the inner stent 1505 relative to the outer stent 1507 are applicable to various stent architectures, including, but not limited to, braided stents and laser cut stents such as z-stents.

One or more coupling points 1530 may be employed to secure the inner and outer stents 1505 and 1507. The holes 1506 may also circumferentially extend about the distal ends of the stents 1505 and 1507 such that multiple coupling points 1530 are created. Generally speaking, utilizing a greater number of coupling points 1530 will increase the degree to which inner stent 1505 is coupled to the outer stent 1507. The exact number of coupling points 1530 to be utilized will depend at least in part on the target site for deployment and the size of the target site. For example, if the stent-within-a-stent arrangement is to be deployed within a body lumen such as the esophagus which undergoes peristalsis, multiple coupling locations may be desired so as to maintain the inner stent 1505 in a predetermined fixed location within outer stent 1507. If the stent-within-a-stent arrangement is to be deployed within a relatively smaller body lumen such as the biliary duct which does not undergo frequent peristalsis, a single coupling location 1530 may be sufficient to couple the inner and outer stents 1505 and 1507 without significantly increasing the delivery profile of the stent-within-a-stent arrangement. Although not shown, the proximal-most struts of the inner and outer stents 1505 and 1507 may also contain holes into which the cannula 1501 may be secured thereto. Furthermore, although the location of the coupling points is shown to occur at one or both ends of the stents 1505 and 1507, the location of the coupling points 1530 may also occur along the body portion of the stents 1505 and 1507.

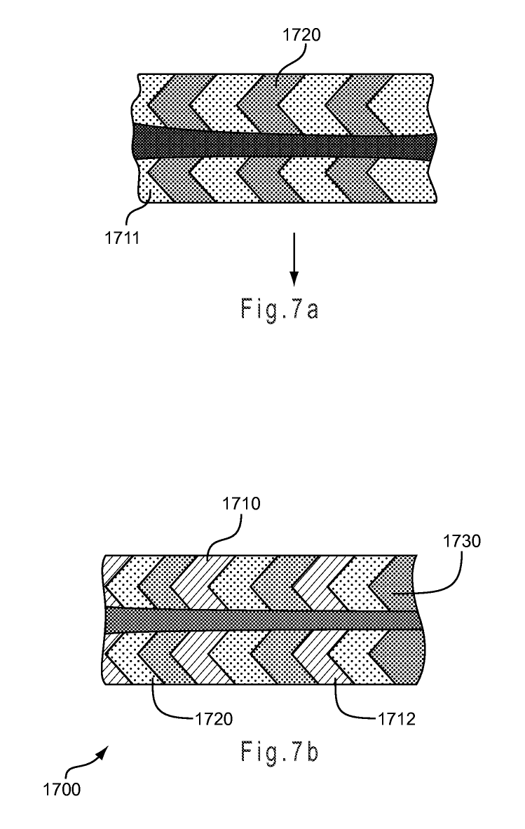

If the inner stent and the outer stent have the same helical pitch, then the inner stent may be disposed slightly offset from the outer stent to create the arrangement shown in FIG. 17b. FIGS. 7a and 7b are partial cross-sectional views through the walls of their respective stents. FIG. 7b shows an inner z-stent 1710 disposed slightly offset from an outer z-stent 1720 to create a stent-within-stent arrangement 1700. The struts 1712 of inner z-stent are positioned offset from the struts 1730 of outer z-stent 1730. FIG. 7a shows interstices 1711 of outer z-stent 1720 in which no inner stent 1710 has been inserted therewithin. Upon deployment of inner z-stent 1710 into the lumen of outer z-stent 1720 (as indicated by the arrow below FIG. 7a), the interstices 1711 may decrease by about 50% relative to the interstices 1711 in FIG. 7a.

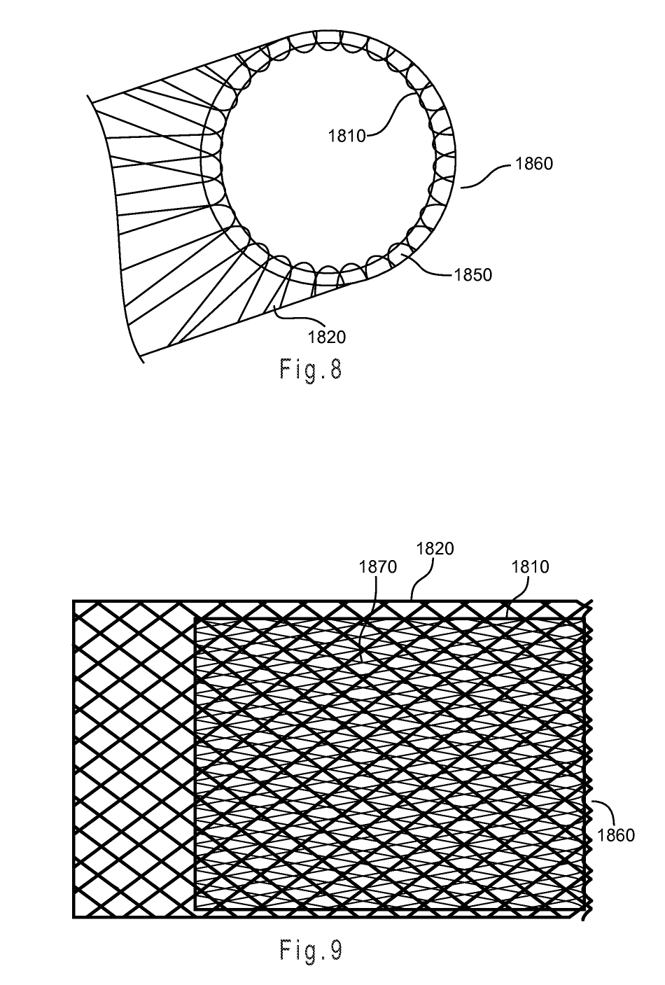

FIGS. 8 and 9 show another embodiment for maintaining a stent-within-stent arrangement. The contribution of radial force provided by the inner stent 1810 may be sufficient to prevent the inadvertent migration of the inner stent 1810 from the lumen of the outer stent 1820. However, as an additional safety feature, FIGS. 8 and 9 show that inwardly folded crowns 1850 along the distal end 1860 of outer stent 1820 may function to prevent the inner stent 1810 from migrating completely outside from the lumen of the outer stent 1820 at the target site, as clearly seen in FIG. 9. In particular, the distal-most crowns 1850 of the outer stent abut against the struts 1870 of the inner stent 1810 to prevent the inner stent 1810 from further distally sliding out of the lumen of the outer stent 1820. FIG. 8 shows that the apices of the crowns 1850 are folded inwardly into the lumen of the inner stent 1810, thereby causing the crowns 1850 to abut against the struts of inner stent 1810. Preferably, the crowns 1850 are folded inwards 90° or greater relative to the wall of the outer stent 1820. Having inwardly folded crowns 1850 only along the distal end 1860 of the outer stent 1820 may be preferred when the inner stent 1810 has a tendency to migrate distally, as could occur when the inner and outer stents 1810 and 1820 are deployed within the esophageal region.

Although all of the distal crowns 1850 are shown bent inwardly, only a portion of the distal crowns 1850 may be bent inwards so as to abut the struts of the inner stent 1810 and prevent further distal movement of the inner stent 1810 from the lumen of the outer stent 1820.

Preferably, the inner stent 1810 is configured within the outer stent 1820 so as to extend the length of the stenosed region to prevent tissue ingrowth through the interstices of the outer stent 1820. The outer stent 1820 is preferably formed from a shape memory material. Tissue-ingrowth is permitted to occur along the ends of the outer stent 1820 because of the absence of struts 1870 of the inner stent 1810 occupying the interstices of the outer stent 1820 along either end thereof. The tissue-ingrowth through the ends of the outer stent 1820 may sufficiently anchor the outer stent 1820 at the target site within the body lumen.

Alternatively, an outer stent 1820 with flanged ends, or any other type of end portion having an outward radial force sufficient to prevent migration, may provide sufficient anchorage of the outer stent 1820 at the target site without the need for tissue ingrowth through interstices of the outer stent 1820 to provide the necessary anchorage. Accordingly, an inner stent 1810 extending the entire length of the outer stent 1820 can be deployed within the lumen of such an outer stent 1820 capable of providing sufficient anchorage at the ends thereof.

In another embodiment, the inner stent 1810 may expand to a diameter equal to or greater than the expanded diameter of the outer stent 1820 so as to impart a radial force outwardly against the interior surface of outer stent 1820. The contribution of radial force by inner stent 1810 may be sufficient to anchor the stent-within-stent arrangement such that tissue ingrowth through the outer stent 1820 ends and/or reliance on end portions of outer stent 1820 (e.g., flanged ends) capable of providing sufficient anchorage are not required.

Still referring to FIGS. 8 and 9, the crowns along the proximal end (not shown) of the outer stent 1820 remain parallel to the longitudinal axis of the outer stent 1820, thereby enabling the inner stent 1810 to be inserted into the lumen of the outer stent 1820 from the proximal end of the outer stent 1820. The inner stent 1810 is not anchored within the lumen of the outer stent 1820. To prevent inadvertent migration of the inner stent 1810 from within the lumen of the outer stent, FIGS. 8 and 9 show that the outer stent 1820 may have crowns 1850 along the distal end that revert from a parallel configuration to an inwardly folded configuration after deployment at a target site as a result of the shape memory properties of the outer stent 1820. Alternatively, the distal crowns 1850 of the outer stent 1820 as shown in FIGS. 8 and 9 could be pre-formed into the inwardly bent shape, thereby eliminating the need for the crowns 1850 of the stent 1820 to be formed from a shape memory material capable of moving from a parallel to bent orientation.

Alternatively, the inner stent 1810 may contain crowns along one or both ends thereof that revert from a parallel configuration during delivery to an outwardly folded configuration after deployment at a target site as a result of the shape memory properties of the inner stent 1810. The proximal and distal crowns of the inner stent 1820 would preferably be designed to flare outwardly to engage the struts of the outer stent 1810, thereby fixating the inner stent 1810 relative to the outer stent 1820 within the lumen of the outer stent 1820. Preferably, the crowns of the inner stent 1810 flare outwards a sufficient amount to engage and abut against the struts of the outer stent 1820 while not perforating any tissue through the interstices of the outer stent 1810.

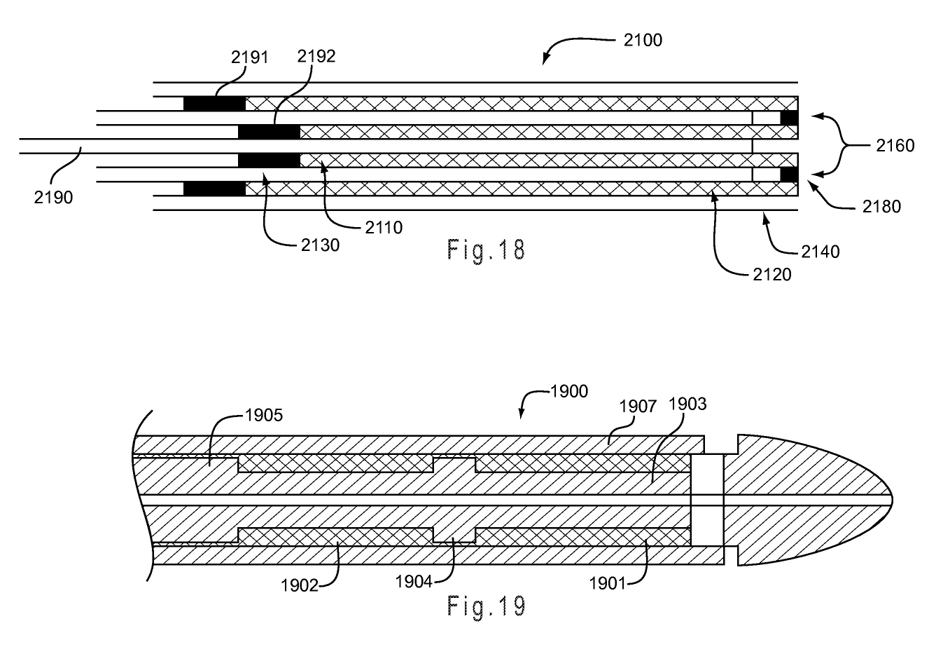

The shape memory material from which the crowns 1850 may be formed is preferably a nickel-titanium alloy. The temperature memory of the nickel-titanium alloy causes the crowns 1850 to move from a parallel configuration during delivery to the folded configuration (FIGS. 18 and 19) after deployment. Specifically, the nickel-titanium alloy crowns 1850 may undergo a transformation between a lower temperature martensitic phase and a higher temperature austenitic phase. The delivery configuration of the crowns 1850 comprises the martensitic phase of the nickel-titanium alloy. The deployment configuration of the crowns 1850 comprises the austenitic phase of the shape memory material. Austenite is characteristically the stronger phase, and martensite may be deformed up to a recoverable strain of about 8%. Strain introduced into the crowns in the martensitic phase to achieve the parallel delivery configuration of the crowns may be recovered upon completion of a reverse phase transformation to austenite, allowing the crowns 1850 to return to a previously-defined inwardly folded or outwardly folded shape (the deployment configuration). The forward and reverse phase transformations may be driven by application and removal of stress (superelastic effect) and/or by a change in temperature (shape memory effect). According to an alternative embodiment, the parallel delivery configuration of the crowns may comprise the austenitic phase and the deployed inwardly/outwardly flared configuration of the crowns may comprise the martensitic phase. When using temperature induced memory, it is preferable that the nickel-titanium alloy has a transformation temperature which is less than or equal to the body temperature (37° C.) so that transformation to the austentic phase is triggered when the crowns 1850 are positioned at the target site.

FIG. 18 shows that a single introducer 2100 may used to deploy the inner and outer stents 2110 and 2120 described above. The inner and outer stents 2110 and 2120 are shown constrained within their respective delivery sheaths 2130 and 2140. FIG. 18 shows that the inner and outer stents 2110 and 2120 are coupled with radiopaque markers 2160 at distal end 2180.

Although not shown, anchors or crowns as described above may be used on either the inner stent 2110 or outer stent 2120. During delivery, such anchors or crowns are preferably oriented parallel to the longitudinal axis of the sheaths 2130 and 2140 to avoid frictional resistance between the sheaths 2130 and 2140 and the anchors or crowns.

The single introducer 2100 may be advantageous over conventional introducers because it maintains separation of the stents 2110 and 2120 during delivery within their respective sheaths 2130 and 2140, thereby preventing inadvertent entanglement of the struts of the inner and outer stents 2110 and 2120. In use, with the stents 2110 and 2120 in their loaded configuration as shown in FIG. 18, the single introducer 2100 is advanced to the target site. Upon reaching the target site, the outer sheath 2140 is retracted in the proximal direction relative to the central inner catheter 2190, thereby deploying the outer stent 2120. Stopper 2191 prevents the outer stent 2120 from being pulled back with its respective outer sheath 2140. At this juncture, the inner stent 2110 remains coupled to the outer stent 2120 but not yet deployed. Sheath 2130 is retracted in the proximal direction relative to the inner catheter 2190 to deploy the inner stent 2110 within the lumen of the outer stent 2120. Stopper 2192 prevents inner stent 2110 from being pulled back with its respective sheath 2130. Visualization of the inner stent 2110 and the outer stent 2120 relative to the target site is possible via the radiopaque markers 2160 at distal end 2180.

Although the inner and outer stents 2110 and 2120 are shown coupled at their respective distal ends, the stents may be loaded into the single introducer 2100 in their noncoupled state, as previously described. Rather than deploy the stents 2110 and 2120 simultaneously, the stents 2110 and 2120 would be deployed one at a time. The outer stent 2120 would be deployed by retracting outer sheath 2140 followed by deployment of the inner stent 2110 by retracting sheath 2130. Having the inner stent and outer stent 2110 and 2120 decoupled within the single introducer 2100 during delivery allows placement of the inner stent 2110 within a specific location of the lumen of the outer stent 2120. In other words, the configuration of the inner and the outer stents 2110 and 2120 in their loaded state within the single introducer 2100 may be substantially the same configuration the inner and the outer stents 2110 and 2120 attain in their deployed state.

Additionally, the single introducer 2100 of FIG. 18 may be used in connection with a conventional expandable member, such as a balloon catheter, for purposes of dilating the body lumen and setting the position of the first stent 1901 and/or the second stent 1902, as known in the art. The additional dilation force may enhance fixation of the first stent 1901 and/or the second stent 1902 into the tissue at the target site.

It should be understood that the inner and outer decoupled stents may also be deployed simultaneously using a conventional introducer in which the inner stent is disposed within the lumen of the outer stent. Upon proximal retraction of the outer sheath relative to the inner catheter, both the inner stent and the outer stent are simultaneously deployed at the target site.

FIG. 19 shows an alternative single introducer 1900 that may be used to deploy an inner stent within an outer stent as has been described above. FIG. 19 shows the introducer 1900 serially loaded with a first stent 1901 and a second stent 1902. The second stent 1902 is shown proximally spaced apart from the first stent 1901. Each of the first and the second stents 1901 and 1902 are mounted onto a pusher member 1903. The pusher member 1903 has a first shoulder 1904 engageable with the proximal end of the first stent 1901 and the distal end of the second stent 1902. The first shoulder 1904 may maintain separation of the first stent 1901 from the second stent 1902 during advancement of the stent-loaded introducer 1900 to a target site. The pusher member 1903 also has a second shoulder 1905 engageable with the second stent 1902. The second shoulder 1905 engages with the proximal end of the second stent 1902 when the pusher member is distally advanced relative to the outer sheath 1907 to remove the second stent 1902 from within the introducer 1900. The introducer 1900 may also comprise an expandable member (e.g., balloon catheter) that can be used to dilate the body lumen and thereafter set the position of the first stent 1901 and/or the second stent 1902, as known in the art. Alternatively, the single introducer 1900 could be modified such that a separate expandable member is disposed within the lumen of each of the first stent 1901 and the second stent 1902 when the stents 1901 and 1902 are balloon expandable.

The method of implanting a stent-within-a-stent arrangement in which the inner and outer stents are deployed separately using a conventional delivery sheath will now be described. Referring to FIG. 1b, an outer stent 100 is first delivered and deployed to a target site of a body lumen. The outer stent 100 is allowed to radially expand at the target site. FIG. 1b shows that the outer stent 100 has mesh openings 112 and struts 111 that form the mesh design. After the outer stent 100 is fully deployed, the inner stent 110 may be delivered and deployed within the outer stent 100. As FIG. 1a shows, the inner stent 110 has two anchors 130, 140 that are flush with the surface of the inner stent 110 in the longitudinal direction. Configuring the anchors 130, 140 flush with the inner stent 110 during delivery helps to maintain a low delivery profile that can be constrained within a delivery catheter 120. Because the helical pitch of the inner stent 110 is greater than that of the outer stent 100, the ends of the inner stent 110 need not be offset relative to the outer stent 100. Rather, the ends of the inner stent 110 will be deployed within the outer stent 100 such that its ends are aligned with the ends of the outer stent 100.

The delivery catheter 120 is moved into the radially expanded outer stent 100. At this juncture, the inner stent 110 is partially deployed. The outer sheath of the delivery catheter 120 is slightly retracted to allow the distal end of the stent 110 and the anchors 130, 140 to be exposed. The distal end of the inner stent 110 begins to radially expand. After the anchors 130, 140 and distal end of the inner stent 110 have been exposed from the delivery sheath of the catheter 120, the delivery catheter 120 may be moved around to further manipulate the distal end of inner stent 110 so that the anchors 130, 140 interlock with the outer stent 100 at the desired position. At this point, the anchors 130, 140 may be moved to the interlocking position as shown in FIG. 2. The interlocking position consists of the anchors 130, 140 flaring or bending outwards through the interstices 112 of outer stent and thereafter catching on the struts 111 of the outer stent 100 to secure the inner stent 110 with the outer stent 100. If the anchors 130, 140 are formed from a shape memory alloy such as nitinol, then the anchors may be heat activated or stress activated to revert to the interlocking position.

After each of the anchors 130, 140 have been moved to its respective interlocking position, the entire delivery sheath may be retracted to allow the balance of the inner stent 110 to radially self-expand against the inner surface of the outer stent 100. In this example, because the diameter of the inner stent 110 is about the same as that of the outer stent 100, the inner stent 110 is adequately fitted against the outer stent 100.

If the outer stent 100 and inner stent 110 have identical helical pitches, then the inner stent may be positioned offset relative to the outer stent 100 such that the struts of the inner stent 110 occupy the free spaces 112 or open meshes of the outer stent 100.

Although the above procedure has been described with respect to self-expandable stents, the stents may be balloon expandable. Additionally, any type of stent architectural pattern is contemplated, including, but not limited to, a zigzag, sinusoidal, or serpentine configuration of struts. Any type of laser cut stent pattern is also contemplated.

Deploying individual stents to create a stent-within-stent arrangement as described above eliminates the need to deploy expandable stents with a covering along the body portion. Typically, stents with coverings have delivery profiles which are too large to fit through an accessory channel of an endoscope, thereby making tissue ingrowth a potentially severe problem. Additionally, the tissue ingrowth through the openings of the end portions of the stent may be so severe as to permanently anchor the covered stent at the target site such that removal of the covered stent is not possible. On the contrary, the deployment of an outer bare metallic stent followed by deployment of a bare metallic inner stent as described can solve tissue ingrowth problems while still enabling delivery through an accessory channel and subsequent removal of the outer and inner stents from the target site.

Other advantages in addition to the substantial elimination of tissue in-growth may be achieved using the above-described stent arrangements. For example, replacement of an occluded inner stent with a new inner stent may prolong the life and the patency of the outer stent. Generally speaking, the inner stent acts to protect the interior surface of the outer stent. The inner stent may longitudinally extend only along the length of the stenosed region so as to allow tissue ingrowth through the ends of the outer stent to anchor the outer stent at the target site, if the outer stent is not required to be removed from the body lumen. Removal of the occluded inner stent is possible because tissue in-growth does not occur through the interstices of the inner stent. Alternatively, if an outer stent with flanged ends or other suitable end portion structure is used that exerts a sufficient outward radial force against the walls of the body lumen to provide fixation therewithin, the inner stent may extend the entire length of the outer stent, as the need for tissue ingrowth to provide anchorage is not required. However, an outer stent with flanged ends may not be needed if the inner stent sufficiently contributes to the outward radial force such that no migration of the stent-within-stent arrangement occurs. The inner stent may be anchored to the outer stent with shape memory anchors described and illustrated in FIGS. 1-6. Upon removal of the occluded inner stent, the anchors may be temperature or pressure activated to revert to the parallel martensitic delivery configuration to decouple the inner stent from the outer stent.

Alternatively, it should be understood that various other stent arrangements are contemplated that will prolong the patency of the outer stent. As an example, the inner stent as shown and described above in all of the embodiments may be substituted with a sleeve. FIG. 12 shows a cross sectional view of a braided stent 1100 that contains a removable sleeve 1110 disposed within the lumen and along the interior surface of the outer stent 1100. The sleeve 1110 may be formed from any biocompatible material. The sleeve 1110 may extend along the length of the stenosed region as shown in FIG. 12, thereby allowing tissue ingrowth at the ends 1120 and 1130 of the outer stent 1100 to provide necessary anchorage. Alternatively, if an outer stent with flanged ends or other suitable end portion structure is used that exerts a sufficient outward radial force against the walls of the body lumen to provide fixation therewithin, the sleeve 1110 may extend the entire length of the outer stent, as the need for tissue ingrowth to provide anchorage is not required.

Still referring to FIG. 12, the sleeve 1110 may be coupled to the anchored stent 1100 with shape memory anchors 1150 and 1160. Similar to the inner stents described in the previous embodiments, the inner sleeve 1110 upon occlusion is designed to be removable because it is not permanently anchored to the tissue at the target site. The shape memory anchors 1150 and 1160, which are affixed to the sleeve 1110, may be temperature activated (e.g., cold water or cold saline solution may be injected onto the surface of the anchors 1150 and 1160 to reduce temperature of the anchors 1150 and 1160 below body temperature). The anchors 1150 and 1160 revert to the parallel martensitic delivery configuration to enable decoupling of the inner sleeve 1110 from the outer stent 1100. A retrieval member such as forceps may then be introduced to hook onto one of the anchors 1150 and 1160 and thereafter withdraw the sleeve 1110 from the lumen of the outer stent 1100. After removal of sleeve 1110, a new sleeve can be secured to the outer lumen of outer stent 1100. Accordingly, the inner sleeve 1110 is replaceable, thereby prolonging the patency of the outer stent 1100.

Preferably, the inner sleeve 1110 is substantially nonporous. Accordingly, the inner sleeve 1110 serves as a protective inner covering or sheath over the interior surface of the outer stent 1100 when implanted at the target site. Alternatively, the inner sleeve 1110 with anchors 1150 and 1160 may be formed from biodegradable material that biodegrades at a predetermined time, thereby eliminating the need to remove the inner sleeve 1110. Preferably, the inner sleeve 1110 is designed to begin biodegradation after being occluded. After the inner sleeve 1110 has completely biodegraded, a new sleeve may be deployed, if necessary, within the outer lumen of the outer stent 1100.

Still other advantages in addition to increased patency and reduced tissue endothelialization are contemplated by the above-described stent arrangements. For example, the inner stent may contribute to the overall outward radial force of the outer stent. FIG. 13 shows an embodiment in which a coiled inner stent 1210 is disposed within the lumen of an outer z-stent 1220 to create a stent-within-stent arrangement 1200. FIG. 13 shows that the inner coiled stent 1210 may extend the entire longitudinal length of the outer z-stent 1220 so as to impart additional radial force along the entire length of the outer z-stent 1220. FIG. 13 shows that the inner coiled stent 1210 may impart sufficient radial force outwardly such that the stent-within-stent arrangement 1200 remains fixated at a target site. Alternatively, the inner coiled stent 1210 may be shorter in longitudinal length than the outer z-stent 1220 when deployed within the lumen of the outer z-stent 1220 so as to extend only along the stenosed region of the target site. The inner coiled stent 1210 is shown to occupy the interstices of the outer z-stent 1220 so as to reduce tissue in-growth therethrough. The helical pitch of the inner coiled stent 1210 can be varied as needed to occupy more or less interstices of the outer z-stent 1220. Generally speaking, the outer z-stent 1220 may comprise any type of stent architecture. Preferably, the inner stent is a foreshortening stent, such as the coiled stent 1210 shown in FIG. 13, in which there is a reduction in the diameter associated with a corresponding increase in the length of the inner stent when pulling on an end of the inner stent during its retrieval from the lumen of the outer stent. As a result, such foreshortening stents may facilitate removal of the inner stent from the lumen of the outer stent. Removal of the inner coiled stent 1210 may occur if an occlusion lodges into the lumen of the inner coiled stent 1210.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A medical device for dilation of a body lumen, comprising:
an expandable outer prosthesis formed from a plurality of outer struts, each of the plurality of outer struts being spaced apart to form outer openings therebetween; and
an expandable inner prosthesis formed from a plurality of inner struts, each of the plurality of inner struts being spaced apart to form a plurality of inner openings therebetween, wherein the inner prosthesis is disposed within a portion of a lumen of the outer prosthesis so that a portion of the inner struts at least partially block the outer openings.

2. The medical device of claim 1, wherein the inner prosthesis has a greater helical pitch than the outer prosthesis so as to define inner openings smaller in size than the outer openings of the outer prosthesis.

3. The medical device of claim 1, wherein the plurality of outer struts define an outer structure and the plurality of inner struts define a plurality of inner structure different from the outer structure.

4. The medical device of claim 1, wherein the inner prosthesis is disposed offset from the outer prosthesis.

5. The medical device of claim 5, wherein the engagement member comprises a shape memory anchor affixed to one of the outer prosthesis and the protective inner prosthesis.

6. The medical device of claim 1, wherein the inner stent in a first expanded state comprises a bent crown flared outwardly a sufficient amount to removably engage with one of the plurality of struts of the outer prosthesis in a second expanded state.

7. The medical device of claim 1, wherein the outer prosthesis in a first expanded state comprises a bent crown flared inwardly a sufficient amount to removably engage with a strut of the inner stent in a second expanded state.

8. A medical device for dilation of a body lumen, comprising:
an outer stent comprising outer struts spaced apart to form outer spaces therebetween;
an inner stent comprising inner struts spaced apart to form inner spaces therebetween, wherein at least a portion of the inner stent is slidably interfitted within the lumen of the outer stent; and
an interlocking element fixating the inner stent within the outer stent, wherein at least a portion of the inner struts cover the outer spaces of the outer struts to substantially prevent tissue in-growth therethrough.

9. The device of claim 8, wherein the interlocking element comprises one or more anchors.

10. The device of claim 9, wherein the one or more anchors are affixed to a surface of at least one of the inner struts and the outer struts.

11. The device of claim 9, wherein the one or more anchors are formed from a shape memory material, the one or more anchors movable between a first configuration and a second configuration.

12. The device of claim 11, wherein the one or more anchors in the first configuration is oriented substantially parallel to a longitudinal axis of the medical device.

13. The device of claim 11, wherein the one or more anchors in the second configuration is bent away from a longitudinal axis of the medical device.

14. The device of claim 8, wherein the interlocking element comprises a weld or a magnetic coupling point between the inner stent and outer stent.

15. The device of claim 8, wherein the interlocking element comprises a cannula extending through a hole of the inner struts and the outer struts.

16. A method of implanting a stent arrangement into a body lumen, comprising the steps of:
(a) delivering an outer stent and an inner stent to the body lumen;
(b) deploying the outer stent and the inner stent at a target site within the body lumen, the outer stent expanding from a first diameter to a second diameter greater than the first diameter, the outer stent having a plurality of outer struts spaced apart at the second diameter to form a plurality of outer openings; and
(c) interlocking the inner stent to the outer stent.

17. The method of claim 16, wherein the interlocking step comprises securing one or more shape memory anchors of the inner stent to a strut of the outer stent by moving the one or more anchors from a first configuration during delivery to a second configuration at deployment, the first configuration being parallel to a longitudinal axis of the inner stent, and the second configuration being flared outwardly a sufficient amount to interlock with a strut of the outer stent.

18. The method of claim 16, wherein the interlocking step comprises securing one or more shape memory anchors of the outer stent to the inner stent by moving the one or more anchors from a first configuration during delivery to a second configuration at deployment, the first configuration being parallel to a longitudinal axis of the outer stent, and the second configuration being flared inwardly a sufficient amount to interlock with the inner stent.

19. The method of claim 16, further comprising the steps of
(d) bending a crown of the outer stent; and
(e) engaging the bent crown with a strut of the inner stent to prevent migration of the inner stent from the lumen of the outer stent.

20. The method of claim 16, wherein the step of delivering the outer stent and the inner stent comprises loading the outer stent and the inner stent within a single introducer.

21. The method of claim 20, wherein the step of deploying the outer stent and the inner stent further comprises the steps of retracting a first sheath of the single introducer to deploy the outer stent and retracting a second sheath within the lumen of the deployed outer stent to deploy the inner stent therewithin.

22. The method of claim 16, wherein the outer stent and the inner stent are coupled to each other with a cannula prior to delivery at the body lumen.
